(19) United States
(12) Patent Application Publication
(10) Pub. No.: US 2022/0142859 A1
(43) Pub. Date: May 12, 2022

(54) A TIME SAVING SIT ON CARDIO PULMONARY RESUSCITATION DEVICE AND METHOD

(71) Applicant: Krishna Prasad Panduranga Revankar, Bangalore (IN)

(72) Inventor: Krishna Prasad Panduranga Revankar, Bangalore (IN)

(21) Appl. No.: 17/433,469

(22) PCT Filed: Feb. 25, 2020
(86) PCT No.: PCT/IN2020/000007

§ 371 Date: Aug. 24, 2021

(30) Foreign Application Priority Data
Feb. 26, 2019 (IN) 201941007405

(51) Int. Cl. A61H 31/00; A61N 1/39; A61M 16/06; A61M 5/42; A61M 1/00; A61M 16/20; A61M 5/20; A61M 16/00

(52) U.S. Cl. CPC A61H 31/006; A61M 2005/206; A61M 16/0622; A61M 5/427; A61M 5/425; A61M 1/87; A61M 16/202; A61M 16/0683; A61M 5/20; A61M 16/0057; A61H 31/007; A61H 2201/107; A61H 2201/1207; A61H 2201/1246; A61H 2201/1253; A61H 2201/5071; A61H 2201/5058; A61H 2230/045; A61H 2201/10; A61H 2201/105; A61H 2201/5064; A61M 2205/3313; A61M 2210/02; A61M 2205/3553; A61M 2205/502; A61M 2205/3327; A61N 1/39044

(57) ABSTRACT

A time saving sit on cardio pulmonary resuscitation device and method wherein the said device for providing cpr is adopted with an arrangement to seat a person and enable the start of cpr within five minutes of a heart attack affecting a patient, comprising of a reciprocating resuscitation force applicator where a counter force to the reactive force arising on applying compression force for resuscitation, said counter force being provided by the weight of a person sitting on the seating means and a belt based drive conveyance means forming a loop from top of a enclosure box allowing cpr without latching. A very clearly understandable, unambiguous, two step method of sitting on the device placed around the patient body, no confusion, no decision steps, no mental thinking on what to do. The device takes care of most decisions automatically.

### CLAIMS OF PRIORITY

This patent application claims priority from the Provisional Patent Application No. 201941007405 filed on 26 Feb. 2019.

### BACKGROUND OF INVENTION

The present invention is a device, system, and method of cardiopulmonary resuscitation hence forth called cpr, which is an important first aid method to help survival of a persons who have suffered a cardiac arrest and is given wide publicity and training, however the survival rate is dismally poor around 2 percent, even though machines to aid the chest compression process in cpr are there in prior art and in market.

However, these machines are not popular due to a serious disadvantage they have in starting the cpr process, as the circulation of blood must be restored to the brain within 5 minutes of blood circulation stoppage to prevent permanent brain damage. The present invention overcomes this disadvantage with inventive steps and a new method of starting cpr along with various other advancements to increase the survival rate of the person who has suffered cardiac arrest hence forth referred to a patient. By stander generally referred to as first responder or as a by passing rescuer, whose help is critical and there is hesitance of men providing resuscitation to women and vice versa, this invention overcomes this hesitancy factor.

### FIELD OF TECHNOLOGY

This disclosure relates generally to technical fields of a medical device used in emergency medicine to resuscitate a person who has suffered a heart attack with lack of blood circulation particularly designed to be used by a bystander. Prior art has inventions related to cpr devices and methods with mechanical reciprocating compression systems developed as early as 1965 taught in patents such as U.S. Pat. No. 3,509,899 C. E. Hewson 1970, Heart and lung resuscitator, U.S. Pat. No. 3,364,924 C. E. Barkalow Pneumatically operated closed chest cardiac compressor, 1968, U.S. Pat. No. 4,196,725, Arthur. M. Gunderson, Cardio pulmonary resuscitation apparatus ,1980, WO2014057116A1 WIK, lars et al, 2012, Chest compression device, US 2014/0005578A1 Pierre. H. Woerlee et al, 2014, Automated CPR device, U.S. Pat. No. 7,226,427 Stig Steen, Lund, Lucas T M, Jolife A. B, Sweden, 2003, System and procedure for treating cardiac arrest, U.S. Pat. No. 8,690,804B2 Anders Nilsson et al, 2014, CPR Apparatus and method, U.S. Pat. No. 2006/0264789A1 Kenneth. H. Mollenauer et al, 2006, Resuscitation device and method, U.S. Pat. No. 9,532,924 B2 Uday kumar V. Illindala et al, 2017, Chest compression devices for use with imaging systems and methods of use with imaging systems, U.S. Pat. No. 5,222,491 Samuel D. Thomas, 1993, Temporary patient ventilator, US 2015/0173598 A1, Mark Alexander, 2015, Intubating Airway, U.S. Pat. No. 10,10,688 B2, Chunyaun Qui, 2018, Intubation system and methods based on airway pattern identification, US 2018/0169426 A1, Gary Montague et al, 2018, Automatic external defibrillator device and method of use, US2005/0049504A1, Meng Tsung Lo et al, 2005, Ultrasonic vein detector and related methods. A user manual of Lucas™ and Zoll™ devices is available in public domain and indicates how the prior art device and method works in practice.

Prior art has severe shortcomings by the very method of unpacking the prior art device and proper attachment of the prior art device to the patient and then start the resuscitation process. The time available to start blood circulation is less than 5 minutes, there is no time left for normal human thinking process after the initial shock and surprise in the sudden event of a heart attack. Bystander help is very critical, there is no time to educate or give him expertly instructions. Due to these difficulties in prior art the success rate of cardiopulmonary resuscitation is dismally low at 2 percent. A manicure-based training of general public and even medical professionals are not widely popular and has its own difficulty to remember steps and sequence, position needed for the rescuer and the correct amount of pressure to be applied. Prior art devices need correct latching to provide counter force for the compressive element and also for correct positioning of compression pad. This is where time is lost for the first-time user, figuring out how to use and what to do wastes valuable time, time to overcome shock and surprise, time to unpack the device, figuring out where the buttons to start is present, wastes even more time. It may be easy to latch but will take time to figure out how to latch for a new user. Inserting the prior art belt drive-based device under the patient, wastes critical time and needs another person's help.

The present invention and method addresses all the above problems with an automated mechanical pressure compression source with sitting arrangement being a well-supported and prominent seat to provide a counterweight for compression strokes. A latching member being one of a pad, a plate and a belt with coupler henceforth called as back pad, that is inserted later once the circulation is started, with the latching member being suspended from the arm or leg of the device and further a patient carrying board is also inserted much latter, below the patient, to enable transport of the patient.

### SUMMARY OF INVENTION

A device, system and method to save time to start cardiopulmonary resuscitation (cpr) without a need for fixing a belt, encircling around a patient's body, immediately to allow trust forces for compression in the resuscitation apparatus, to act on the patient, with the said cpr device having a seat with supporting arms or legs, hence forth referred to as arm which stands for arm or a leg with said arm or leg being a supporting element for the seat and an enclosure box for its associate's mechanism, with said seat meant to provide a counter force to the reactive force arising on application of the compression force, which is provided by the weight of the body of a rescuing person who sits on the seat and once cardiopulmonary resuscitation process is started and when other rescuers help has arrived, fixes the retaining back pad after which the said rescuer can get up from the seat. The enclosure box for the device mechanism may be adopted or modified to form a seat and having supporting arms, in one embodiment.

A sit on device to give required counter force to the reactive force arising on application of the compression force henceforth referred to as counter force for brevity and convenience, with the said counter force being otherwise given as in prior art, by correct strapping and fixing or latching of device to patients' body, which however, in a real life situation, there is not enough time to do correct strapping or latching, by an untrained person.

A device for providing cpr adopted with an arrangement to seat a person and enable start of cpr within 5 minutes of a heart attack affecting patient who has collapsed and comprising of a reciprocating resuscitation force applicator, the said sitting arrangement is to provide a counter force for the resuscitation force being provided by the weight of a person with the said device comprising predominantly a seating means to provide counter weight with seat having supporting arms, with the said reciprocating resuscitation force refers to compression of chest of the patient and after that expansion of chest by pulling up the compression pad, said arm having flanges which provides electrical insulation for rescuer from ground, when a defibrillation shock is applied.

Importantly a drive conveyance means comprising a belt forming a loop at one of a pulley, a roller on a rod attached to arms on the underside of seat and one of a loop around a rod attached to arms, with the said drive conveyance means, helps avoid the need for strapping the belt around patient's body. The above said belt may be a chain, a rope, a cable, or a wire henceforth referred to collectively as a belt.

Further the said resuscitation device comprises of a seat having supporting arm with a slot or slit to insert a plate, a pad, or a belt hence forth referred to as a back pad, a sidebar at lower end of arms to hold the back pad, support for seat being a support arm, a compressor pad adjustment means to correctly locate position or place on chest to apply pressure, referred to as adjustable compression pad, a drive for the compression force being selected from a group of pneumatic, electric motor, electric solenoid based, mechanical and manual force with the drive being conveyed by one of a belt, which as mentioned earlier also refers to a rope, a cable, and a chain, and further an insulated carrier of electrodes for defibrillator and ecg measurement, air supply pump giving positive air pressure means for ventilation with an air mask and a pillow, a microprocessor to control the compressor, a point of compression on patients body, read and analyze ecg, ultrasound signal, send alert for help, along with a programmed instruction set to control and coordinate resuscitation process, the said micro controller to time the spring release by activating solenoid triggers. A communication means to central server to alert emergency medical team to shift patient for reestablishing circulation. A mobile communication means which is started to give instructions where necessary to the rescuer by medical experts and a hands-free microphone and speaker placed on the enclosure box.

A mechanical override is provided in case of automatic system failure or battery exhaustion, a subsystem to help a rescuer to locate the place where the device is kept. A prominent heart sign board is associated with a press button to start a siren indicator on the device, wherein on pressing button wirelessly actuate a siren on the device. Siren and other sound producing means to indicate location of device and help rescuer to locate the device. An apparatus locator alarm which goes on when words like help, that is a word actuated and mobile app actuated alarm and a voice activation by a speaker of Amazon, google like alexa to pick up words like heart attack, help, shouting, to start the process to indicate location of the device by emitting location indicating sound to a rescuer. A device presence indicator having stickers that glow in dark, a light emitting diode flashers and beepers, a central electrical bus bar to connect to any available power source, an extra battery bank, the support arm of seat having provision to hold battery pack. Air pump to provide ventilation along with pure oxygen can. and a head tilt pillow with air mask and an oxygen cylinder and supplementary cylinders.

The device further comprises of a defibrillator attachment with water repellence with forced air jet in case patient has fallen in rainy weather or in a bathroom where ground is wet wherein the electrode being mounted on a frame which pops out and pops in from enclosure box and pushed down by a spring to contact the patient's body, the said electrodes being connected to lead wires of ecg, defibrillator and ultrasound transducer receiver, and further wherein an insulating sheet is provided to protect rescuer when applying defibrillator current, and further since instruction giving defibrillator can confuse the rescuer so the defibrillation process is automated and a sound based detection of tachycardia by sensor and fibrillation detected by multiple means by heart beat amplifier and speaker, further an adhesive is added to defibrillator electrode. along with a salt solution to enable conductivity through a cloth like a shirt and having additionally a safety cloth cutter with suction cup to lift up a cloth and cut with safety cutter enclosed blade in said safety cutter, and in one embodiment, the arm support has attached defibrillator electrode, and one of the electrode is fixed on the belt and compression pad.

A simple version, with minimum features, of an echocardiogram based and ultra sound based imaging from transducer and sensor is attached to the device, to guide the resuscitation process and intubation and the device further having a pulse oximeter on compression pad and also as separate attachment. An insulator sheet having multiple electrodes, so that required electrode is chosen to analysis by the micro controller which may also be called as a computer or a microprocessor, and a blood pressure gauge and pulse oximeter is also provided in advanced version of the device.

Pre attached latching points on a cot for hospital use as an adopter to cot of hospital bed for quick attachment of device and a system of cantilever based or a cable based, device delivery to the patient location. As a further advancement to the basic version of the device an automated vein locator and injector of life saving drug like clot buster is provided.

A battery backup and electric cord or a cable to connect to a electric power source from an electric grid to continue resuscitation till required. Above device used in beaches and swimming pools being delivered using drones and having additional attachment to suck out water from the lung. A rain protection sheet is also provided and as further improvement a high capacity electrical capacitor for solenoid based and motor-based compressor is provided.

A program app to alert a person to go near a resuscitation device location, and take help of available people wherein ecg electrodes attached to a person help in detecting early changes in heart activity before a heart attack.

The system accommodates all fuzzy work done by an untrained person and make suitable corrections. The method of providing cpr using a rescuers body as counterweight may be even by standing on or keeping one leg on the device.

Up and down movement of compressor pad to start automatically, initially with less force to test check, and after detecting that a patient is not conscious by means of sensors or by the rescuer, with device starting program in micro controller automatically when a person sits on the seat and when a person's presence in between the arms, is detected by proximity sensors.

A self-attachment method is used wherein patient alerted of impending heart attack by sensors and warning devices, can attach the device themselves and wait until help arrives.

### Other Important Features

A more advanced version of the resuscitation device wherein an automatic intubation devise is present, is described in the description of drawings. Auto intubation device to puncture cricoid membrane to get airway, which is blocked. An automatic vein locating attachment is provided and if vein is not located a drill attachment to drill hole in the bone to reach bone marrow to inject required clot busting drugs is provided.

In a further embodiment the device is evolved to form a chair having a resuscitation device with belt drive and straps, with chair having prominent heart sign for identification in an emergency. with chair having distinctive color and visible flagging to indicate its presence even from a distant, and being present in workplaces offices, software companies, and said chair having seat height adjustment facility and caster wheels, and a chair based simple ventilator. This is not a mere combination of a two know devices as the combination gives a different function of avoiding latching, saving time to start cpr, whereas the prior art independent devices cannot give this functionality.

In a further embodiment a telescopic arm for backpack version for use in field and outdoor locations is provided and for affordability reasons have a simple manual operation. Two rods otherwise used as support, toggle up to enable manual application of force in case the powered system does not work for any reason.

The device starts by test compressions with lower pressure to correct position of pressure point and assess patient condition like lung compliance and build up pressure to required level after verifying correct positioning of pad or pressure point and a simple spring-based switch to detect correct compression along with load sensor, with said load sensor being present in the enclosure box at seat end rather than on plunger.

### SCOPE OF INVENTION

A death due to heart attack as seen in case of prominent persons like A P J Kalam could have been prevented if a timely action as enabled by the present system was present and seeing a politician collapse and fall down is not an excuse specially when intelligent ways and systems as that taught by the present invention can be deployed in large numbers. The cultural and moral problem of providing cardiopulmonary resuscitation to a woman, a lady who is a stranger, in a public place is a real problem. similarly women will hesitate to provide resuscitation to a man and by the time a decision by overcoming inhibition happens, it's too late to save a life. This invention perfectly solves this ethical dilemma enabling anyone to help anyone else. The lost lives so far is an evidence for non-obviousness of the present invention.

Emergency handling in hospital can be greatly improved so doctors can come more relaxed and need not run, this will revolutionize emergency medicine. A very clearly understandable, unambiguous two step method of sitting on the device placed over the patient, no confusion, no decision steps, no mental thinking on what to do. Even keeping a skilled and trained person being available all 24 hours for a very important person like presidents and ministers etc is a challenge so the need for whoever available person to provide help in case of a cardiac event.

Single person can start and gain time, getting a second persons to help wastes time in telling him or her what to do, communication wastes time and leads to decision errors.

The device, systems, and methods disclosed herein may be implemented in any means for achieving various aspects, and may be executed in a form of a machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF DRAWINGS

FIG. 1 and FIG. 2 depicts a rescuer performing cpr on a male patient on a street and on a female patient respectively.

FIGS. 3 and 4 show the patient and rescuer as in FIGS. 1 and 2 with air mask attached.

FIG. 5 a preferred embodiment of a resuscitation device, which is based on a compressor belt-based drive conveyance means.

FIG. 6 an embodiment of device, which is based on pneumatic drive.

FIG. 7 is a sectional view of device with compressor pad holder to adjust correct point on sternum of a patient, to apply compression pressure.

FIG. 8 is a view of the x-y coordinate movement means for the compressor pad.

FIG. 9 a schematic and simplified view of spring-based drive means.

FIGS. 10 A and 10B is a simplified view of a manually operated cpr device.

FIG. 11 is another embodiment of a cpr device where a rescuer 1101 applies counter force by standing on a step 1102 provided to the arm of the device.

FIG. 12 is an embodiment of a cpr device where a rescuer 1201 applies counter force by standing on one of the step 1203 and a rod 1202, placed on the other step.

FIG. 13 is an embodiment of a cpr device where a rescuer 1301 applies counterforce by means of a handle 1302 by leaning on the said handle.

FIG. 14 is a view of an embodiment of a cpr device 1401 showing a support for the seat of device being a side arm 1402.

FIG. 15 a view of an embodiment of a cpr device 1501 showing a support for the seat of device being a telescopic leg 1502.

FIG. 16 an embodiment of a cpr device 1601 modified into a chair.

FIG. 17 is a simplified view of an extra attachment provided in a more advanced version of the cpr device, with the attachment being an automatic incubation unit 1701.

FIG. 18 is a simplified view of an extra attachment provided in a more advanced version of the cpr device, with the attachment being an automatic vein detection and drug injection unit 1801.

FIG. 19 a view of the pop out frame carrying ecg and defibrillator electrodes.

FIGS. 20 TO 27 are drawings of how the prior art device is used as given in the user manual of Lucas™ device, while FIGS. 28 to 31 are drawings of how the prior art device is used as given in the user manual of Zoll™ device. These drawings are given to explain the time delay to start cpr as happening in prior art devices. FIGS. 32 a, b, c, 33a, b, c and 34 are schematic drawings, specially given to avoid any confusion on what is the counter force to the reactive force arising on applying compression force and why the counter force provided by body weight of rescuer is needed or necessary to avoid latching or fixing of device to patent body.

### DETAILED DESRIPTION OF DRAWINGS

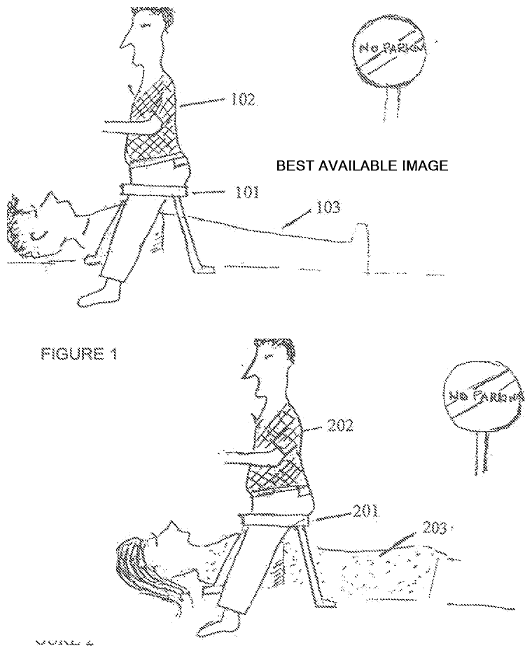

FIG. 1 a depiction of a rescuer performing cpr on a male patient on a street with resuscitation device being 101, a rescuer 102, and a patient 103, a no parking sign board is shown to indicate the place as being a street.

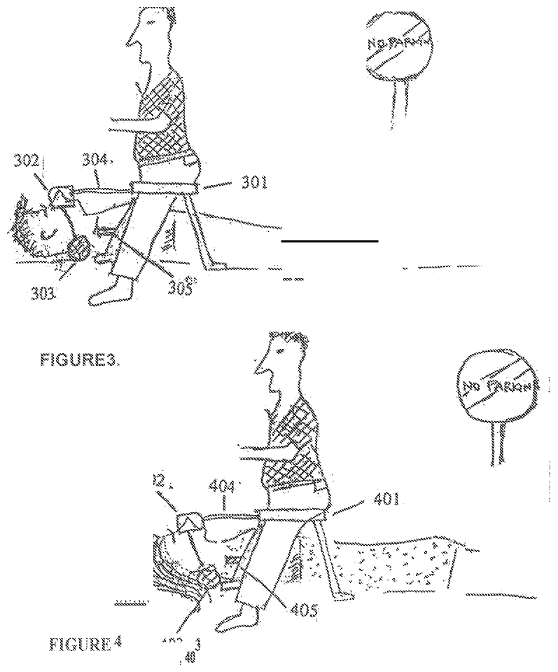

FIG. 2 a rescuer performing cpr on a female patient on a street with resuscitation device being 201, a rescuer 202, and a patient 203, the rescuer is not hesitant as he may be, with manual resuscitation on an unknown lady in a public place.

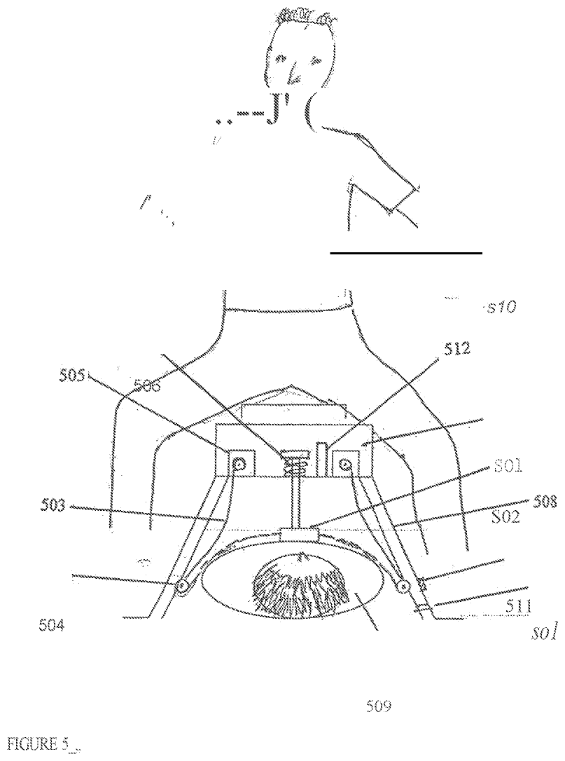

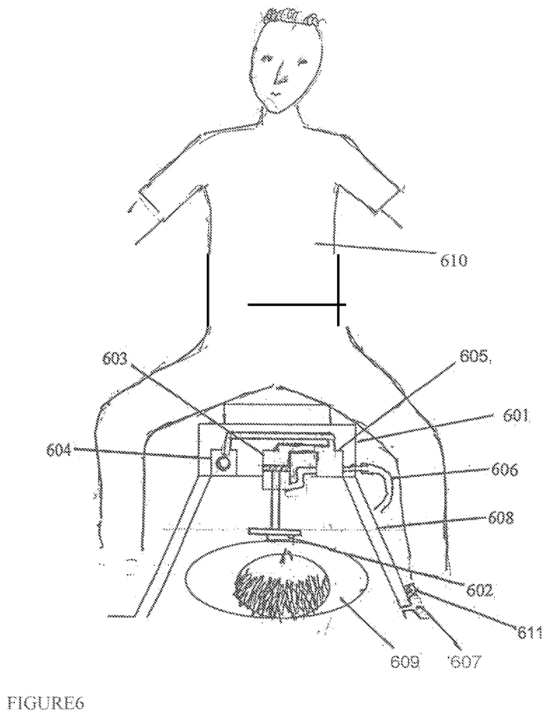

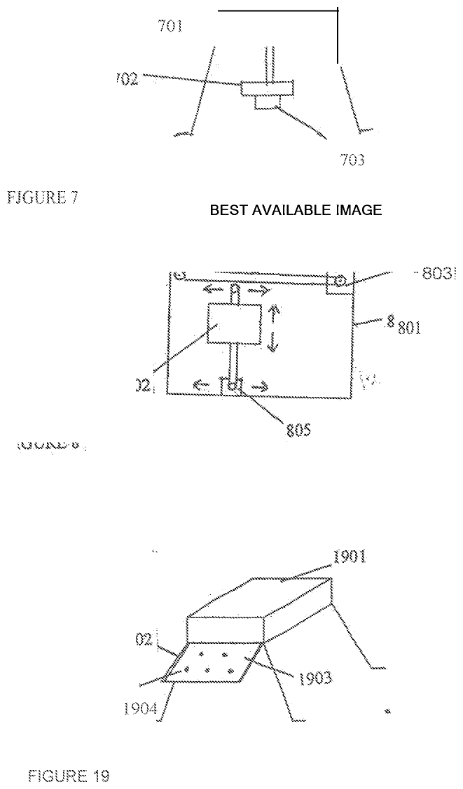

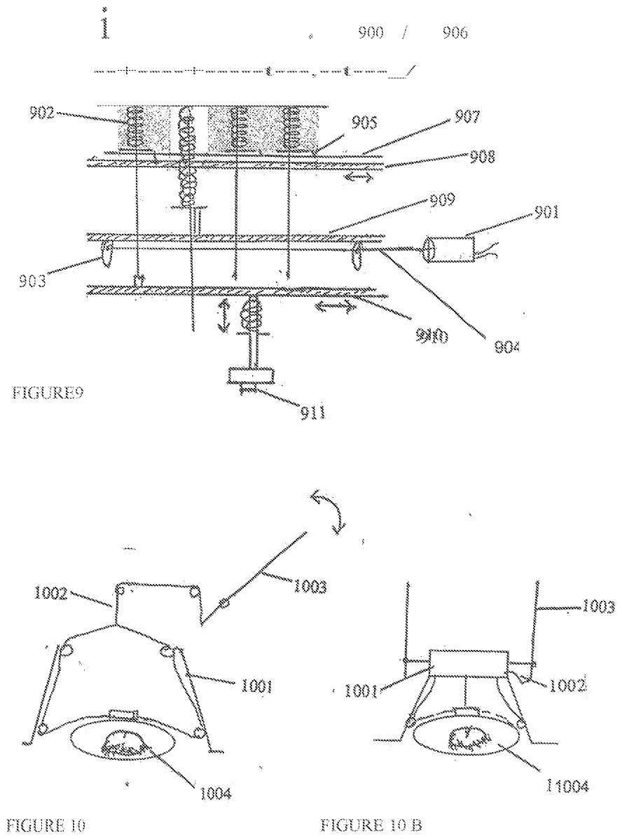

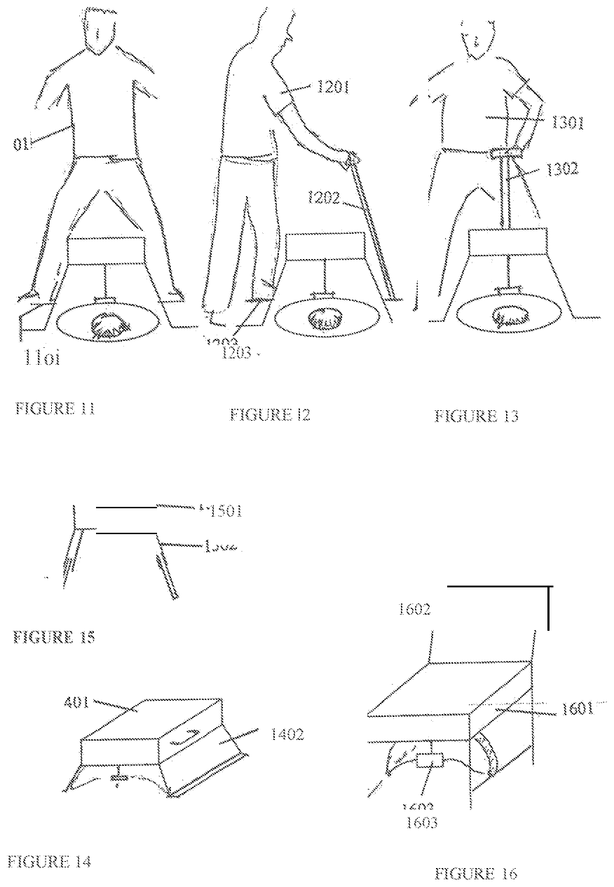

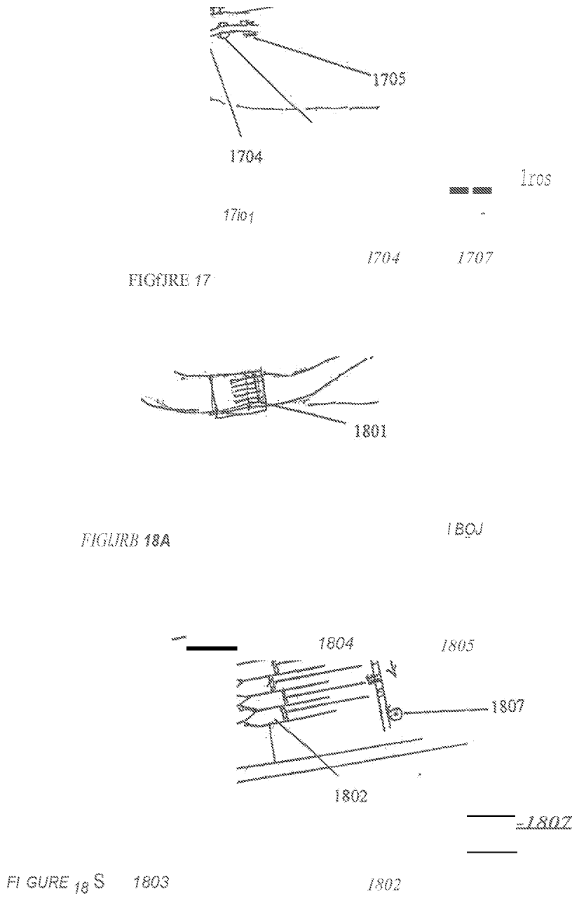

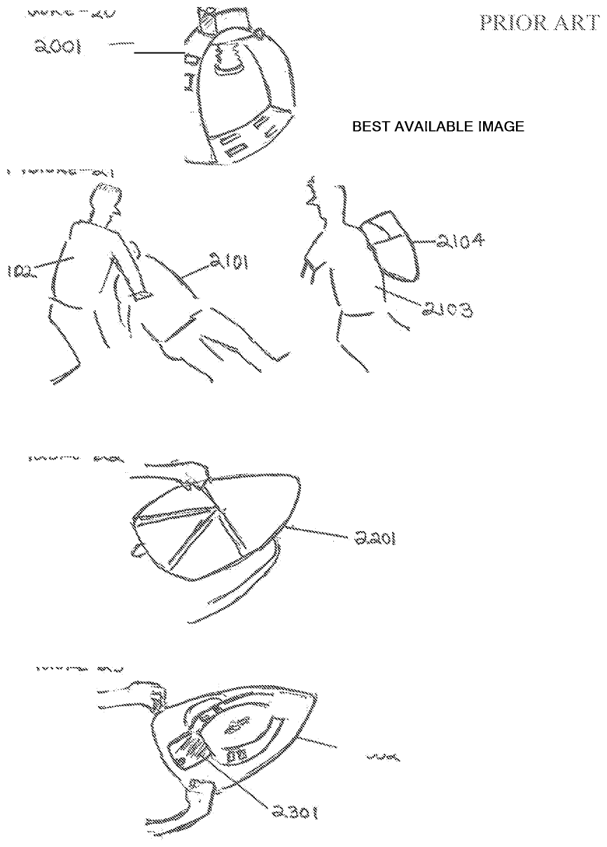

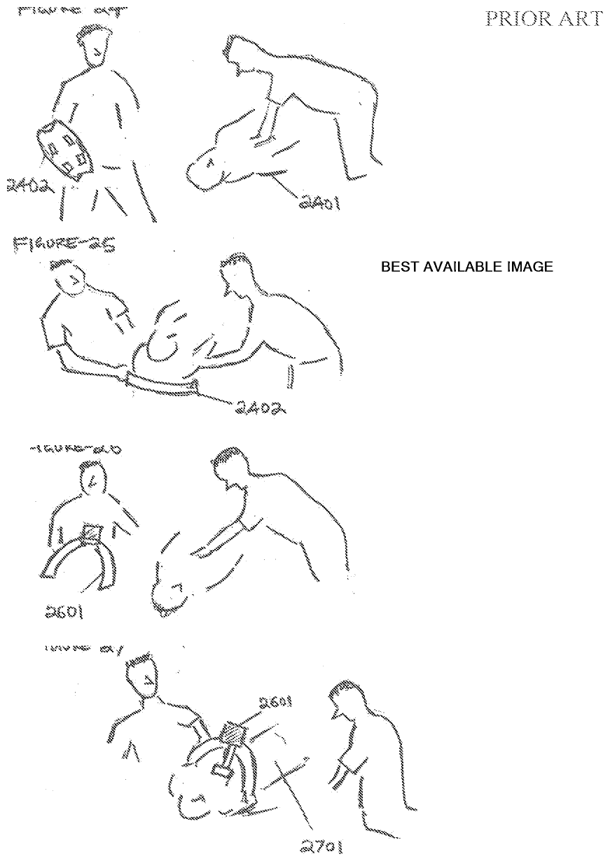

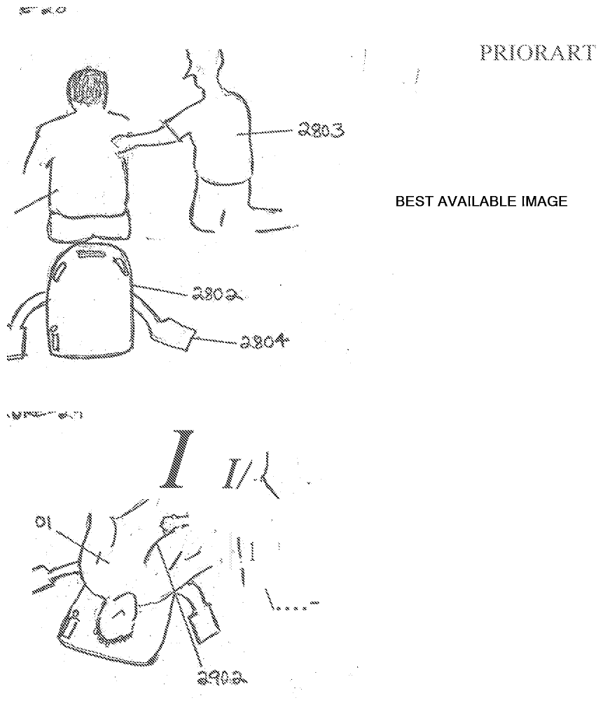

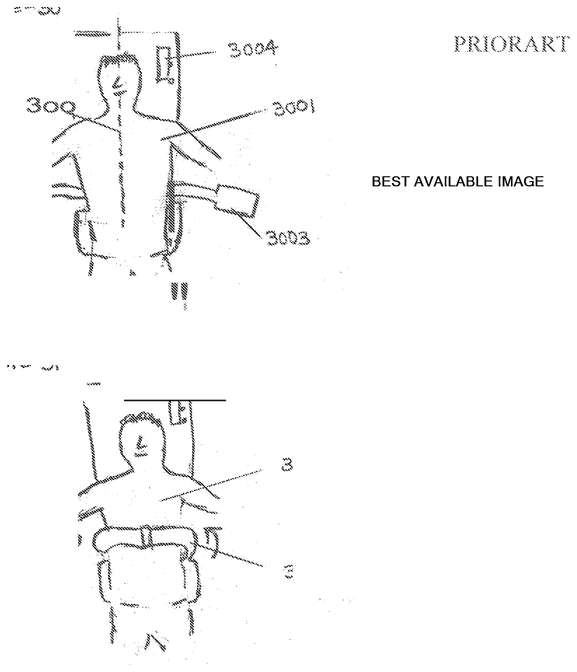

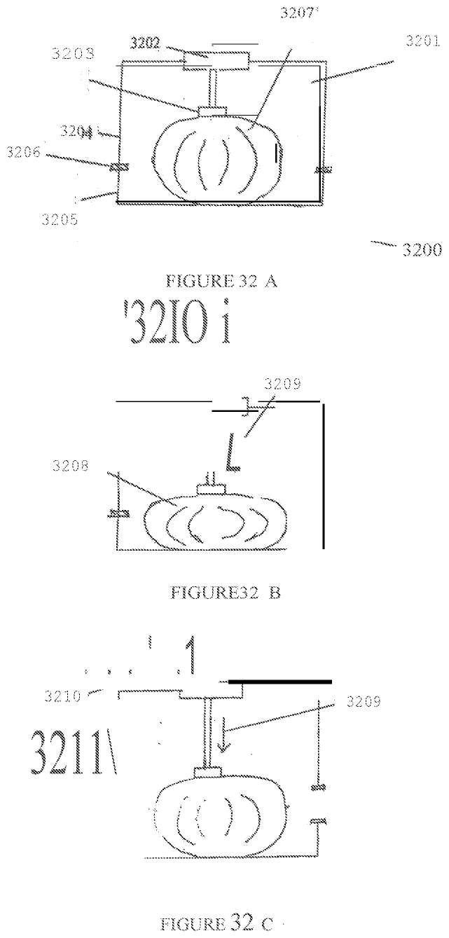

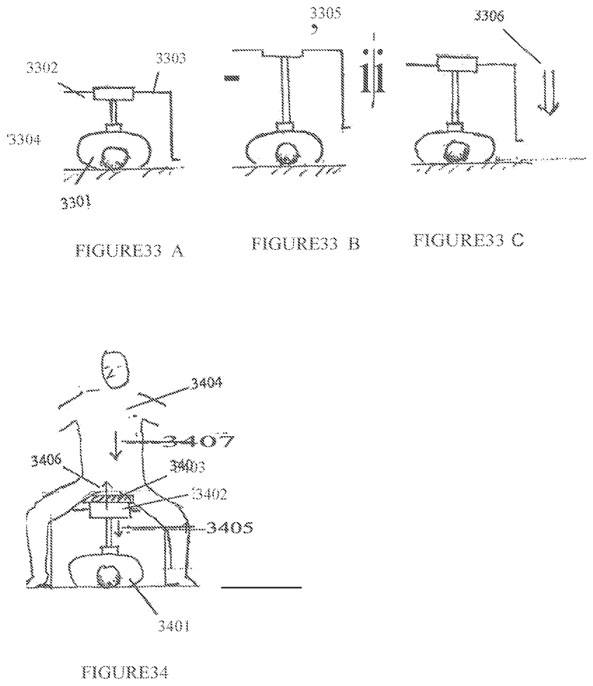

FIG. 3 the patient and rescuer as in FIG. 1 with air mask 302, with air pipe 304 attached to resuscitation device 301, a neck tilt pillow 303 and a defibrillator electrode 305.

FIG. 4 the patient and rescuer as in FIG. 2 with air mask 402, with air pipe 404 attached to resuscitation device 401, a neck tilt providing pillow 403 and a defibrillator electrode 405.

FIG. 5 a preferred embodiment of a resuscitation device which is a compressor belt based, with device being 501, having compressor pad 502, compressor belt 503 passing through belt guide pulley 504 and belt being driven by electric motor with spool 505, compressor pad return spring 506, leg or arm 508 having slot 507 for inserting retain board or belt referred to as back pad, under a patient to secure the patient for transit once circulation is established, with patient being 509 and rescuer 510, defibrillator electrode holder 511 and air pump for ventilation 512.

FIG. 6 an embodiment of a resuscitation device being 601, having compressor pad 602, pneumatic cylinder with piston being 603, air compressor pump with electric motor 604, pressurized air storage tank 605, pipe with connector for external compressed air supply 606, leg or arm 608 having slot 607 for inserting retain board or belt under a patient once circulation is established, with patient being 609 and rescuer 610, a defibrillator electrode holder 611.

FIG. 7 is a sectional view of the resuscitation device 701, with a compressor pad holder 702, and a compressor pad 703, whose position can be adjusted to correct point on sternum of a patient, to apply compression pressure.

FIG. 8 is a view of the adjustable compression pad, having a x and y coordinate movement means for the compressor pad 802, on compressor pad holder 801, belt 804 driven by a fixed electric motor 803 for movement in x direction, and a moving electric motor drive 805 for movement in y direction of the said compressor pad 802.

A position of compression point adjustment means provides an adjustment means for the correct point of compression on a patient's chest, with compressor pad being present on a pad holder, with pad having x-y coordinate movement of compressing member, said movement is driven by belt, moved by electric drive motors.

FIG. 9 a schematic and simplified view of spring-based drive for providing compression force having a electric drive motor 901, a drive shaft 904, a set of springs 902 having rod with notch and ratchet 900, held by frame 906 and 907, with spring being compressed by cams 903 being present on the drive shaft 904 driven by motor 901, a spring engagement plate 909 selects a spring to be compressed, a latch trigger for release of spring being 905 released by action of latch release plate 908, a compression drive engagement plate 910 drives the compression pad 911 to which the springs apply force through a rod 900, the engagement plates being operated by a separate electric motor and cams or by electric solenoid.

FIGS. 10 A and 10B is a simplified view of a manually operated resuscitation device 1001, with belt drive similar to that described in the embodiment of resuscitation device in FIG. 5, with said belt drive being connected to force transfer cable 1002 to receive force on working or movement of rod 1003 by a rescuer, with patient being 1004.

This manual drive means is added to other embodiments as an emergency manual override in case the automatic system, does not work due to any fault.

FIG. 11 is another embodiment of a resuscitation device where a rescuer 1101 applies counter force by standing on a step 1102 provided to leg or arm of the resuscitation device.

FIG. 12 is another embodiment of a resuscitation device where a rescuer 1201 applies counter force by standing on one of the step 1203 provided to leg or arm of the resuscitation device and a rod 1202 placed on the other step.

FIG. 13 is yet another embodiment of a resuscitation device where a rescuer 1301 applies counter force by means of a handle 1302 by leaning on the handle, provided for the resuscitation device while standing.

FIGS. 11, 12, and 13 shows that the cpr device essentially has the counter force provided by the rescuers body. It is a method of providing cpr using a cpr device wherein a counter force to the reactive force for the compression force needed for resuscitation, is provided, and the said counter force being provided by the weight of a person, who is a rescuer.

FIG. 14 is a view of the resuscitation device 1401 showing a support for the seat of device being a side arm 1402 formed of the lid cover of enclosure box and the enclosure box is provided with a handle to help carry the box like a suitcase box.

FIG. 15 a view of the resuscitation device 1501 showing a support for the seat of device being a telescopic leg 1502 with this embodiment being meant to enable carry the device for used in open field by mountain climbers and soldiers.

FIG. 16 an embodiment of the resuscitation device 1601 being built into a regular chair 1602, with compressor pad being 1603, with the said chair 1602 being clearly labeled as a heart chair to be used in emergency while still being used as a regular chair at other times and legs having coulter wheels.

FIG. 17 is a simplified view of an extra attachment provided in a more advanced version of the resuscitation device, with the attachment being an automatic incubation unit 1701, a guide tube 1703 having a intubation tube 1702 which is driven forward by a pinion which engages with a rack present on the wall of the tube, a epiglottis depressing flap 1704 operated by cable present in the tube, a magnetic piece 1705 at near the tip of intubation tube, a probe 1706 outside on neck region to detect position and orientation of intubation tube, correct entry into trachea detected by means of ultrasound reflection analysis and by standing wave formed by sound from a speaker and received by a microphone, thereby avoiding entry into esophagus, and an inflatable balloon 1707 to enable respiration

FIG. 18 A and B is a simplified view of an extra attachment provided in a more advanced version of the resuscitation device, with the attachment being an automatic vein detection and drug injection unit 1801 which is attached around the arm of a patient, with vein position detected by means of infrared image captured by an infrared camera 1808 and ultra sound and reflectance of light from a laser light falling on the skin and, with structural frame holding syringes 1802, having needles 1803 and moved by rack and pinion driven by motor 1804, means to hold a needle on a syringe in a low angle and a drive for the needle along with syringe being a rack and pinion driven by motor 1805, engagement bar 1806 to select required syringe that has connected with a vein, and drive for the engagement bar 1807.

FIG. 19 is a view of the resuscitation device 1901 having a pop out sheet 1903 made of insulating material and having array of electrodes 1904 for ecg and defibrillation with said sheet being held by rods 1902 and the sheet pressed on the chest of patient. A cloth cutting safety cutter is also provided to help attach electrodes automatically.

Additional drawing regarding prior art cpr devices currently being used, are being provided so that the speed advantage occurring from the present invention becomes apparent as an inventive step, only on comparison of prior art user instructions manual, with the present disclosed invention, on how the speed comes about, even though its already described in the main description. The description of additional drawing from the manual of instructions of representative cpr devices of Lucas™ and Zoll™ company, as prior art in order to bring out the main difference in speed of usage between the present invention and prior art inventions.

FIG. 20 view of prior art cpr device 2001 from Lucas chest compression system. FIG. 21 cpr device 2104 being brought to the patient 2101 by person 2103 who is a 2^{nd} rescue worker while the patient is being provided manual cpr by person 2102 as 1^{st} rescuer. FIG. 22 show the cpr device in bag 2201 and FIG. 23 shows the cpr device 2301 inside bag 2302, device being unpacked. FIG. 24 shows the back plate 2402 being taken out while patient 2401 is being given manual cpr. FIG. 25 shows the back plate 2402 being inserted below the patient. FIG. 26 shows the upper part of the Lucas device 2601. FIG. 27 shows the upper part of the device 2601, fixed to the back plate below the body of the patient 2701 and the mechanical cpr started.

FIG. 28 shows the prior art cpr device known as Zoll™, with the rescuer 2803 lifting up the body of the patient 2801 to the sitting position to enable correct placement of the device 2802 having band with pads 2804. FIG. 29 shows the patient 2901 after being placed on the device and rescuer is shown removing the clothing 2902.

FIG. 30 shows the patient 3001 placed and aligned on center line 3002, and the device having band with pads 3003 and control panel display and buttons 3004. FIG. 31 shows the patient 3101 with the band pads 3102 attached and ready for mechanical cpr.

Further additional drawings are provided to clarify about what the counter force to the compression force is, which is a counter force to the reactive force arising in opposite direction to the compression force, with the said reactive force arising at the same time as when the compression force is being applied. This should not be confused with the compression pad and its rods return force provided by spring shown as 506 in FIG. 5 where I have called the said spring 506 as compression pad return spring.

I have tried to explain the counter force to resuscitation force with schematic diagrams for the sake of simplicity with FIG. 32 A showing a schematic diagram of the cpr device being placed on a ground 3200, with the cpr device being 3201, having enclosure box 3202 containing compression drive, a compression pad 3203, front part of frame 3204, connected to back part of frame 3205 having back pad or plate with said connection being by latch 3206, and into this frame a pumpkin 3207 is placed which is acted upon by compression pad 3203.

The compression action by compression pad on the pumpkin is shown in FIG. 3 B with arrow 3209 showing direction of compressive force, arrow 3210 shows direction of the reaction force which is opposite to the compression force and occurring at the same time as the compression force. FIG. 3B shows the compression force acting on the pumpkin resulting in the pumpkin getting compressed; notice the latch 3206 is fixed. Now disconnect the latch 3206 and thereby disconnect front part of frame 3204 from back part of frame 3205 and start the machine, what happens is shown in FIG. 3C The reactive force 3210 moves the front part of the frame, away from compressor pad and the pumpkin is not compressed, since a force to restrain the upper frame from moving is missing when latching is not done. Avoiding latching and fixing is essential to save time to start cpr assuming an untrained, low intelligence bystander has to connect the cpr machine and has to do it with in 3 minutes when the brain damage begins to start from hypoxia, 3 minutes as told to me by a cpr expert. So, when no latching is done the reactive force shown by arrow 3210 has to countered with an oppositely directed force, the said force being external to the device. This counter force is what has been called all along this patent document, as counter force to resuscitation force and this external force is provided by the weight of a rescuing person as described from the beginning to the end of this document.

FIG. 33 A shows the patient 3301 surrounded by the cpr device 3302 with upperpart of frame 3303 having enclosure box which has drive mechanism, and latch disconnected shown as 3304. FIG. 33B shows the front part of frame moving upwards as indicated by arrow 3305 under the influence of reactive force to the compression force while FIG. 33 B shows the front part of frame moving downwards under the force 3306 from weight of device. FIG. 34 shows the patient 3401 with the novel cpr device 3402 with seat 3403 and rescuer 3404 and arrows 3405 showing compression force, 3406 the reactive force and 3407 the counter force to the reaction force of compression force byway of the rescuers body weight.

Although the present embodiments have been described with reference to specific example embodiments, it will be evident that various modifications and changes may be made to these embodiments without departing from the broader spirit and scope of the various embodiments.

In addition, it will be appreciated that the various operations, processes, and methods disclosed herein may be embodied in a machine-readable medium and/or a machine accessible medium compatible with a data processing system (e.g., a computer system), and may be performed in any order (e.g., including using means for achieving the various operations). Accordingly, the specification and drawings are to be regarded in an illustrative rather than a restrictive sense.

I claim:

1. A device for providing cpr adopted with a arrangement to enable start of cpr with in 5 minutes, without wasting time by avoiding latching and fixing of the device to a person at the start of cpr, wherein the said person has collapsed from a heart attack, with the said device comprising of a resuscitation force applicator, the said force applicator having a arrangement to provide a counter force to the reactive force arising on applying compression force for resuscitation, the said counter force being provided by the weight of a person, with the said device comprising
predominantly a seating means being at least one of a seat and one of a enclosure box adopted to allow a person to sit;
supporting arms for the device;
a drive means to provide resuscitation force;
a drive conveyance means;
a face mask with air supply pump and tube;
a micro controller.
The device is characterized by an arrangement to provide the said counter force by using a persons body weight by predominantly a seating means for a person, which is a functional necessity for providing the said counter force, an improved drive means and a micro controller enabling time saving steps.

2. The device as in claim 1
wherein the drive means comprising of a drive force from one of a electric motor, a electric solenoid, along with a battery, its charger and a power chord to connect to a power line, a pneumatic piston drive, a manual mechanical drive, that is by human muscle power, an internal combustion engine drive in combination with initial start by electric motor drive and wherein the drive means is the said manual drive operated with a lever which gives the compression force and the said manual drive is a over ride facility as a redundancy measure in case of a non working of automatic system for any uncontrollable reason and wherein the pneumatic drive is by compressed air from a pump operated by one of a electric motor, one of manually, one of a compressed air cylinder, one of a compressed air produced by chemical reactant, and further an extra electric cable is provided for connecting to a mains power line.

3. The device as in claim 1
wherein the drive means has a further advancement with a arrangement of springs that are charged by the drive force, enabling use of smaller capacity motor and battery of lower current rating by using a set of spring pressing a set of rods with a ratchet and a notch which on release of a latch trigger, push a drive engagement plate connected to rod with compression pad, with said springs being compressed turn by turn by a spring engagement plate, which selects the spring to compress and is driven by cams on a rotating shaft connected to a electric motor, with charged spring released by latch trigger selected by a latch release plate and wherein the said spring engagement plate, the latch release plate and drive engagement plate is one of circular type being revolving and one linear type being translating to and fro, depending on arrangement of spring and wherein in another embodiment the springs are charged by separate motors and released by means of latch operated by solenoid and by an electromagnetic clutch.

4. The device as in claim 1
wherein a drive conveyance means comprises one of a belt drive means comprising one of a belt, rope, chain, a cable passing through one of a pulley and one of a roller, one of a rod forming a bearing surface, located at arm base with the drive means for the belt being at top in enclosure box, avoiding need to lifting a fallen patient to insert device as in prior art, a belt drive from top not requiring any latching, as long as a persons is sitting on the seat, and where in the drive conveyance means is by direct attachment of a drive means to moving rod, said rod being driven by piston of a pneumatic cylinder and by one of a electric solenoid and wherein a retaining back pad to secure device around the patients body being one of a pad, a plate, a belt and a board called as back pad, is provided which is suspended from the arm, allowing rescuer to get up from seat.

5. The device as in claim 1
wherein the adjustable compression pad with position control drive to move the compression pad to the required point on the thorax, being sternum, the compression pad is moved by motor drives in the carrier frame, wherein the required x and y axis movements enable getting the required position, and required position is checked by pressure sensors on the compression pad and with a ultrasound sensor.

6. The device as in claim 1
wherein the device has an enhancement, the said enhancement being a pop out frame carrying electrode to record ecg and provide defibrillation pulse, the frame having a insulated sheet which presses against the chest of a patient and an extra insulation strip to insulate jewels like gold chain and wherein ultrasound transducer and receiver probes are provided to get simple, few parameter signal just enough to help resuscitation.

7. The device as in claim 1
wherein a micro controller using program instructions, starts the presence of device indicating alarm to indicate where the device is located, detects presence of a person by proximity sensor input, and weight of a rescuer activating pressure sensor, and starts the cpr process, detect ecg signal by selecting appropriate electrodes from larger number of electrodes provided and analyse stoppage of heart, detect electrical conductivity of defibrillator electrodes and apply defibrillation current, drives the x y axis motors to give, the required position of compression pad by detecting position on chest using sensor input, controls the latch release of springs in drive means, timing control of compression and air release for air mask tube controlled by solenoid valve and its pressure regulation, communication with rescuer and emergency team started automatically, control the auto intubator motor drive by analysing sensor signals and provide signal to inflate balloon and suck out fluids, display image on monitor to enable the rescuer to correctly intubate, locate vein from ultraviolet image and position control of auto injection and operate selected syringe to administer clot bursting drug, wherein further the micro controller has program steps to monitor and restart resuscitation automatically during transit of a patient to icu if needed and also monitor charging of batteries when device is not in use, analyse ultrasound and echo signals.

8. The device as in claim 1
wherein a nose mask with securing frame and strap and a pillow and connected to air supply from a air pump for providing positive air pressure, provides the required breaths and wherein the said air supply is used for supplying oxygen from a oxygen can.

9. The device as in claim 1
wherein the device as claimed in claim one has an enhancement on the frame which holds the air mask, the enhancement being a automatic intubations means comprising a tube guide and tube pushing rack and pinion, a epiglottis depressing flap, a magnetic guide where a magnet enables a probe outside the body on the neck to detect correct position of the descending or advancing tube with a display screen to monitor insertion into trachea, a inflatable balloon to secure air tube, a separate tube to suck out obstructive or obtrusive fluids, an ultrasound probe on frame placed on neck, further guiding intubation.

10. The device as in claim 1
wherein a further enhancement to the device claimed in claim 1, being an automatic vein locator and injector mounted on a frame, to locate vein by using infrared image sensor base camera, a set of injection needles on a set of syringes, moved to required correct position to engage a vein by rake and pinion, driven by an electric motor, needle with syringe with its plunger moved by another rack and pinion driven by an electric motor, a syringe engagement bar moved by rake and pinion driven by another electric motor to select the syringe with needle that has made a connection with a vein and wherein in case a vein is not connected in time, a drill based injector is provided to drill a bone to access bone marrow to inject drugs to help break thrombus, that is a clot blocking blood flow in a coronary artery.

11. The device as in claim 1
wherein the device has a presence of device indicator, that is its location indicator, the place where the device is stored when not in use, and is informed to a rescuer by an alarm sound and flashing lights and by the microprocessor sending signal to a near by mobile phones to inform location of device and also to alert emergency medical team.

12. The device as in claim 1
wherein a device for providing cpr comprising predominantly a seating means being at least one of a seat and one of a enclosure box adopted to allow a person to sit to provide counter force, supporting arms for the device, and having a drive means being predominantly manual by human muscles driving a rod connected to a lever, a drive conveyance means comprising one of a belt drive means which in turn is comprised of one of a belt, rope, chain, a cable passing through one of a pulley and one of a roller, one of a rod attached to arms forming a bearing surface, said rod located at arm base and with the above said belt being connected to said lever, and further having a face mask with air supply pump.

13. A method of providing cpr using a cpr device wherein a counter force to the reactive force for the compression force needed for resuscitation, is provided, and the said counter force being provided by the weight of a person, who is a rescuer.

14. A device for providing cpr adopted with a arrangement to enable start of cpr with in 5 minutes, without wasting time by avoiding latching and fixing of the device to a person at the start of cpr, wherein the said person has collapsed from a heart attack, with the said device comprising of a resuscitation force applicator, the said force applicator having a arrangement to provide a counter force to the reactive force arising on applying compression force for resuscitation, the said counter force being provided by the weight of a person, with the said device comprising
a enclosure box being selected from one of a enclosure box having its supporting arm provided with a flap, and one of a enclosure box having its supporting arms provided with a step and further selected from a enclosure box provided with a handle for a rescuer to lean on the device, with the said selected enclosure box to enable the application of the said counter force;
a drive means to provide resuscitation force;
a drive conveyance means;
a face mask with air supply pump and tube;
a micro controller.
